(19) United States
(12) Patent Application Publication
(10) Pub. No.: US 2020/0255411 A1
(43) Pub. Date: Aug. 13, 2020

(54) ISOINDOLINE DERIVATIVES

(71) Applicant: VIIV HEALTHCARE UK LIMITED, Brentford, Middlesex (GB)

(72) Inventors: Brian Alvin JOHNS, Research Triangle Park, NC (US); Emile Johann VELTHUISEN, Research Triangle Park, NC (US); Jason Gordon WEATHERHEAD, Research Triangle Park, NC (US)

(21) Appl. No.: 15/776,157

(22) PCT Filed: Dec. 1, 2016
(86) PCT No.: PCT/IB2016/057264

§ 371 Date: May 15, 2018

Related U.S. Application Data
(60) Provisional application No. 62/262,937, filed on Dec. 4, 2015.

(51) Int. Cl. C07D 405/04; A61P 31/18

(52) U.S. Cl. CPC C07D 405/04; A61P 31/18

(57) ABSTRACT

Compounds of Formula I are disclosed and methods of treating viral infections with compositions comprising such compounds.

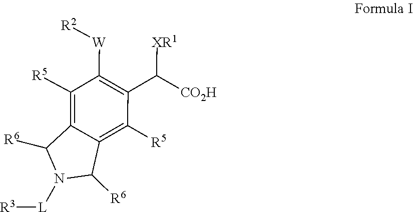

### FIELD OF THE INVENTION

The present invention relates to substituted isoindoline compounds, pharmaceutical compositions, and methods of use thereof for (i) inhibiting HIV replication in a subject infected with HIV, or (ii) treating a subject infected with HIV, by administering such compounds.

### BACKGROUND OF THE INVENTION

Human immunodeficiency virus type 1 (HIV-1) leads to the contraction of acquired immune deficiency disease (AIDS). The number of cases of HIV continues to rise, and currently over twenty-five million individuals worldwide suffer from the virus. Presently, long-term suppression of viral replication with antiretroviral drugs is the only option for treating HIV-1 infection. Indeed, the U.S. Food and Drug Administration has approved twenty-five drugs over six different inhibitor classes, which have been shown to greatly increase patient survival and quality of life. However, additional therapies are still required because of undesirable drug-drug interactions; drug-food interactions; non-adherence to therapy; and drug resistance due to mutation of the enzyme target.

Currently, almost all HIV positive patients are treated with therapeutic regimens of antiretroviral drug combinations termed, highly active antiretroviral therapy (“HAART”). However, HAART therapies are often complex because a combination of different drugs must be administered often daily to the patient to avoid the rapid emergence of drug-resistant HIV-1 variants. Despite the positive impact of HAART on patient survival, drug resistance can still occur. The emergence of multidrug-resistant HIV-1 isolates has serious clinical consequences and must be suppressed with a new drug regimen, known as salvage therapy.

Current guidelines recommend that salvage therapy includes at least two, and preferably three, fully active drugs. Typically, first-line therapies combine three to four drugs targeting the viral enzymes reverse transcriptase and protease. One option for salvage therapy is to administer different combinations of drugs from the same mechanistic class that remain active against the resistant isolates. However, the options for this approach are often limited, as resistant mutations frequently confer broad cross-resistance to different drugs in the same class. Alternative therapeutic strategies have recently become available with the development of fusion, entry, and integrase inhibitors. However, resistance to all three new drug classes has already been reported both in the lab and in patients. Sustained successful treatment of HIV-1-infected patients with antiretroviral drugs will therefore require the continued development of new and improved drugs with new targets and mechanisms of action.

For example, over the last decade HIV inhibitors have been reported to target the protein-protein interaction between HIV-1 integrase and Lens Epithelium Derived Growth Factor/p75 (“LEDGF”). LEDGF is a cellular transcriptional cofactor of HIV-1 integrase that promotes viral integration of reverse transcribed viral cDNA into the host cell's genome by tethering the preintegration complex to the chromatin. Because of its crucial role in the early steps of HIV replication, the interaction between LEDGF and integrase represents another attractive target for HIV drug therapy.

U.S. provisional patent application Ser. No. 62/027,359 discloses certain isoindoline compounds having the following formula:

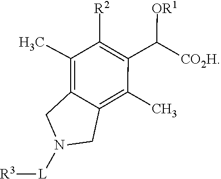

### SUMMARY OF THE INVENTION

Briefly, in one aspect, the present invention discloses compounds of Formula I:

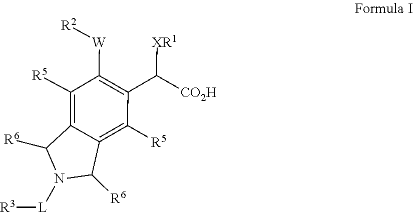

wherein:
X is O or CH₂;
R¹ is C₁₋₆alkyl wherein said alkyl may contain cycloalkyl portions;
W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;
R² is H, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆ alkyl, C₁₋₆ heteroalkyl, or C₁₋₆ alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
L is a bond, —CH₂(CO)—, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;
R³ is H, CN, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, oxo, or R³ may join together with R⁶ or R⁷ to form a fused 5-7 membered ring, and wherein each R³ group is optionally substituted by one to four substituents selected from halo, oxo, C₁₋₆ alkyl, C₃₋₇cycloalkyl, C₁₋₃ fluoroalkyl, —C(O)R⁴, —C(O)NR⁴, —C(O)NHR⁴, C₅₋₁₄aryl, C₁₋₆heteroalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;
R⁴ is CN, halo, —OC₁₋₆alkyl, C₁₋₆ alkyl, C₃₋₇cycloalkyl, C₃₋₉heterocycle, or C₅₋₁₄aryl;
each R⁵ is independently H, C₁₋₃ alkyl, C₃₋₆cycloalkyl, CH₂F, CHF₂, or CF₃;
each R⁶ is independently H, or C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system, with the proviso that at least one R⁶ is other than H;
and wherein each heterocycle, heteroaryl, heteroalkyl, and heteroalkylene comprises one to three heteroatoms selected from S, N, B, or O.

In another aspect the present invention discloses pharmaceutically acceptable salts of the compounds of Formula I.

In another aspect, the present invention discloses pharmaceutical compositions comprising a compound of Formula I or a pharmaceutically acceptable salt thereof.

In another aspect, the present invention discloses a method for treating a viral infection in a patient mediated at least in part by a virus in the retrovirus family of viruses, comprising administering to said patient a composition comprising a compound of Formula I, or a pharmaceutically acceptable salt thereof. In some embodiments, the viral infection is mediated by the HIV virus.

In another aspect, a particular embodiment of the present invention provides a method of treating a subject infected with HIV comprising administering to the subject a therapeutically effective amount of a compound of Formula I, or a pharmaceutically acceptable salt thereof.

In yet another aspect, a particular embodiment of the present invention provides a method of inhibiting progression of HIV infection in a subject at risk for infection with HIV comprising administering to the subject a therapeutically effective amount of a compound of Formula I, or a pharmaceutically acceptable salt thereof. Those and other embodiments are further described in the text that follows.

In accordance with another embodiment of the present invention, there is provided a method for preventing or treating a viral infection in a mammal mediated at least in part by a virus in the retrovirus family of viruses which method comprises administering to a mammal, that has been diagnosed with said viral infection or is at risk of developing said viral infection, a compound as defined in Formula I, wherein said virus is an HIV virus and further comprising administration of a therapeutically effective amount of one or more agents active against an HIV virus, wherein said agent active against the HIV virus is selected from the group consisting of Nucleotide reverse transcriptase inhibitors; Non-nucleotide reverse transcriptase inhibitors; Protease inhibitors; Entry, attachment and fusion inhibitors; Integrase inhibitors; Maturation inhibitors; CXCR4 inhibitors; and CCR5 inhibitors.

### DETAILED DESCRIPTION OF THE INVENTION

Preferably R¹ is C₁₋₆alkyl. Most preferably, R¹ is t-butyl.

Preferably X is O.

Preferably W is a bond.

Preferably R² is optionally substituted phenyl. Most preferably, R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

Preferably R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, C₁₋₃fluoroalkyl, or phenyl.

Preferably each R⁵ is methyl.

Preferably the stereochemistry on the carbon to which XR¹ is bound is as depicted below.

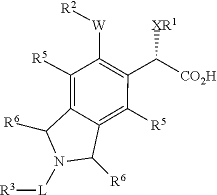

“Pharmaceutically acceptable salt” refers to pharmaceutically acceptable salts derived from a variety of organic and inorganic counter ions well known in the art and include, by way of example only, sodium, potassium, calcium, magnesium, ammonium, and tetraalkylammonium, and when the molecule contains a basic functionality, salts of organic or inorganic acids, such as hydrochloride, hydrobromide, tartrate, mesylate, acetate, maleate, and oxalate. Suitable salts include those described in P. Heinrich Stahl, Camille G. Wermuth (Eds.), Handbook of Pharmaceutical Salts Properties, Selection, and Use; 2002.

### Examples

The compounds of this invention may be made by a variety of methods, including well-known standard synthetic methods. Illustrative general synthetic methods are set out below and then specific compounds of the invention are prepared in the working examples.

The following examples serve to more fully describe the manner of making and using the above-described invention. It is understood that these examples in no way serve to limit the true scope of the invention, but rather are presented for illustrative purposes. In the examples below and the synthetic schemes above, the following abbreviations have the following meanings. If an abbreviation is not defined, it has its generally accepted meaning.
aq.=aqueous
μL=microliters
μM=micromolar
NMR=nuclear magnetic resonance
boc=tert-butoxycarbonyl
br=broad
Cbz=benzyloxycarbonyl
d=doublet
δ=chemical shift
oC=degrees celcius
DCM=dichloromethane
dd=doublet of doublets
DMEM=Dulbeco's Modified Eagle's Medium
DMF=N,N-dimethylformamide
DMSO=dimethylsulfoxide
EtOAc=ethyl acetate
g=gram
h or hr=hours
HCV=hepatitus C virus
HPLC=high performance liquid chromatography
Hz=hertz
IU=International Units
IC₅₀=inhibitory concentration at 50% inhibition
J=coupling constant (given in Hz unless otherwise indicated)
m=multiplet
M=molar
M+H⁺=parent mass spectrum peak plus H+
mg=milligram
min=minutes
mL=milliliter
mM=millimolar
mmol=millimole
MS=mass spectrum
nm=nanomolar
ppm=parts per million
q.s.=sufficient amount
s=singlet
RT=room temperature
sat.=saturated
t=triplet
TFA=trifluoroacetic acid
Z=benzyloxycarbonyl

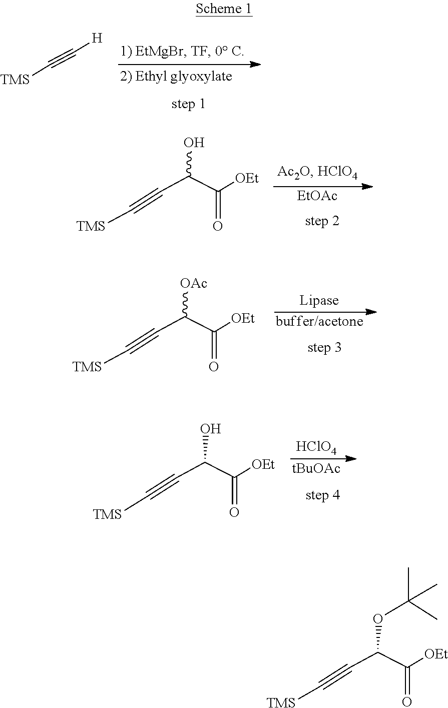

### (S)-Ethyl 2-(tert-butoxy)-4-(trimethylsilyl)but-3-ynoate

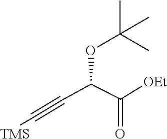 [SMILES: CCOC(=O)[C@H](C#C[Si](C)(C)C)OC(C)(C)C]

### Step 1: Ethyl 2-hydroxy-4-(trimethylsilyl)but-3-ynoate

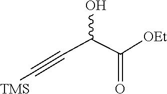 [SMILES: CCOC(=O)C(O)C#C[Si](C)(C)C]

An ice cold solution of TMS-acetylene (250 g, 2.55 mol) in anhydrous THF (2.5 L) was treated dropwise with EtMgBr (933 mL, 2.80 mol, 3.0 M in Et₂O) while maintaining an internal temperature below 5° C. After 30 min, the suspension was cannulated into a flask containing an ice cold solution of ethyl glyoxylate (624 g, 3.05 mol, 50% in toluene) in anhydrous THF (5 L). After 1 h, the reaction mixture was quenched with saturated aqueous NH₄Cl (3 L) and extracted with EtOAc (2×1 L). The combined organics were concentrated at reduced pressure. The residue was diluted with EtOAc (3 L) and washed with water (2×1 L) and brine (2×1 L), dried over Na₂SO₄ and concentrated under reduced pressure. The residue was purified by silica gel chromatography (0-10% EtOAc/petroleum ether) to give the title compound (285 g, 56%) as a yellow oil. ¹H NMR (400 MHz, CHLOROFORM-d) δ=4.83 (d, J=7.3 Hz, 1H), 4.34 (qq, J=7.2, 10.8 Hz, 2H), 3.02 (d, J=7.3 Hz, 1H), 1.34 (t, J=7.2 Hz, 3H), 0.22-0.16 (m, 9H).

### Step 2: Ethyl 2-acetoxy-4-(trimethylsilyl)but-3-ynoate

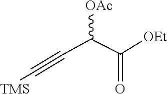 [SMILES: CCOC(=O)C(C#C[Si](C)(C)C)OC(C)=O]

To a 10 L flask was added EtOAc (7.5 L) followed by Ac₂O (400 mL). After stirring at RT for 30 minutes the mixture was cooled to 0° C. and treated with another portion of Ac₂O (2.1 L). After 1 hour at 0° C., the solution was allowed to warm to RT. To the solution was added ethyl 2-hydroxy-4-(trimethylsilyl)but-3-ynoate (520 g, 2.60 mol). After stirring at RT for 1 hour the solution was washed with 1N aqueous NaOH (3×, 20 L total). The solution was then washed with brine (5 L), dried over Na₂SO₄ and concentrated to dryness at reduced pressure. The crude product was purified by flash chromatography (silica gel, 0-5% EtOAc/petroleum ether) to give the title compound (590 g, 94%) as a yellow oil. ¹H NMR (400 MHz, CHLOROFORM-d) δ=5.69 (s, 1H), 4.36-4.21 (m, 2H), 2.19 (s, 3H), 1.32 (t, J=7.2 Hz, 3H), 0.25-0.15 (m, 9H).

### Step 3: (S)-Ethyl 2-hydroxy-4-(trimethylsilyl)but-3-ynoate

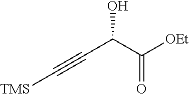 [SMILES: CCOC(=O)[C@@H](O)C#C[Si](C)(C)C]

To a solution of ethyl 2-acetoxy-4-(trimethylsilyl)but-3-ynoate (150 g, 0.620 mol) in acetone (1.88 L) and phosphate buffer solution (pH 7.2, 7.5 L) was added Amano Lipase PS (75 g). After stirring at 20° C. overnight, the reaction mixture was diluted with water (2.5 L) and extracted with EtOAc (3 L). The layers were separated and the organic layer was washed with brine (3×, 10 L total volume), dried over Na₂SO₄, filtered and concentrated under reduced pressure to give the crude product. This material was purified by flash chromatography (silica gel, 0-10% EtOAc/petroleum ether) to afford the title compound (55 g, 44%) as a yellow oil. ¹H NMR (400 MHz, CHLOROFORM-d) δ=4.83 (d, J=7.3 Hz, 1H), 4.34 (qq, J=7.2, 10.8 Hz, 2H), 3.02 (d, J=7.3 Hz, 1H), 1.34 (t, J=7.2 Hz, 3H), 0.22-0.16 (m, 9H).

### Step 4: (S)-Ethyl 2-(tert-butoxy)-4-(trimethylsilyl)but-3-ynoate

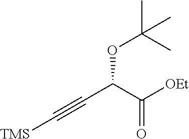 [SMILES: CCOC(=O)[C@H](C#C[Si](C)(C)C)OC(C)(C)C]

To a solution of (S)-ethyl 2-hydroxy-4-(trimethylsilyl)but-3-ynoate (100 g, 0.500 mol) in t-BuOAc (2.5 L) was added HClO₄ (41 mL, 0.500 mol) dropwise at RT. After stirring for 40 minutes, the mixture was quenched with NaHCO₃ powder, diluted with water (2 L) and extracted with EtOAc (2 L). The EtOAc solution was washed with brine, dried over Na₂SO₄, filtered and concentrated under reduced pressure to give the crude product. This material was purified by flash chromatography (silica gel, 0-5% EtOAc/petroleum ether) to afford the title compound (103 g, 81%) as a yellow oil. ¹H NMR (400 MHz, CHLOROFORM-d) δ=4.72 (s, 1H), 4.33-4.20 (m, 2H), 1.31 (t, J=7.2 Hz, 3H), 1.28 (s, 9H), 0.17 (s, 9H).

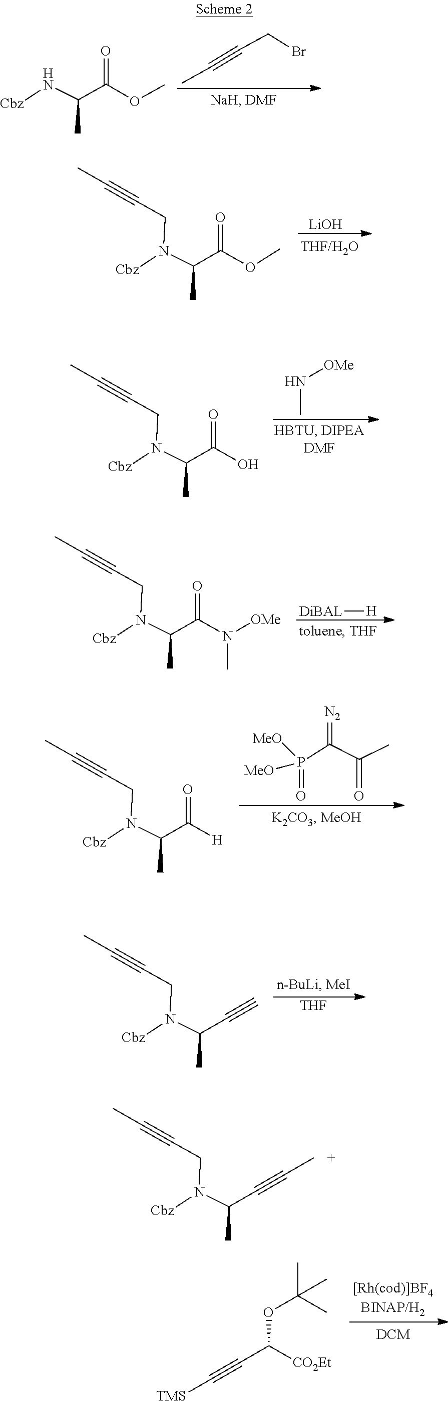

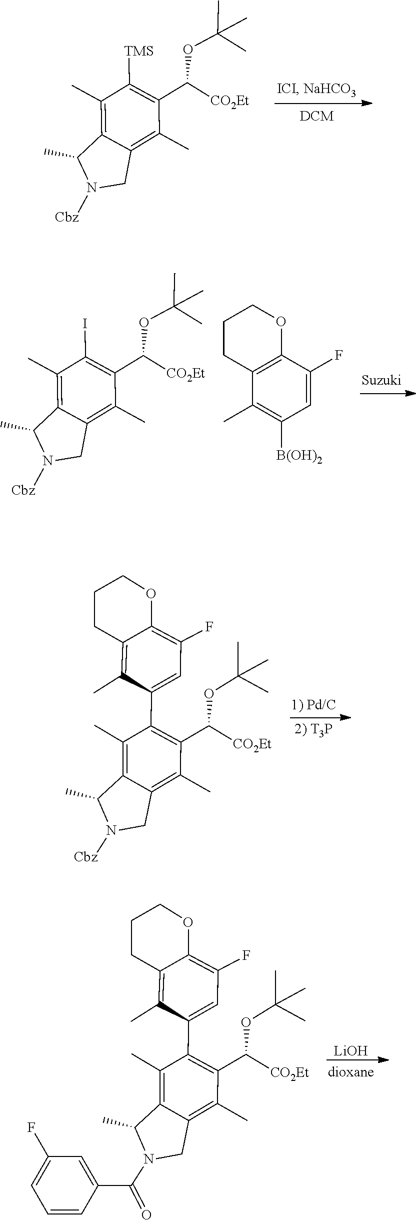

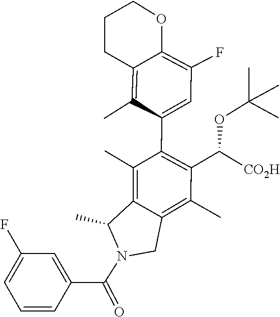

### Example 1: (2S)(M)-2-(tert-butoxy)-2-(-6-(8-fluoro-5-methylchroman-6-yl)-2-(3-fluorobenzoyl)-1-methyl-4,7-bis(trifluoromethyl)isoindolin-5-yl)acetic acid

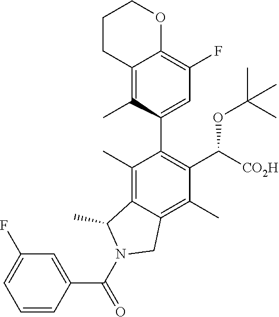

### Step 1: methyl N-((benzyloxy)carbonyl)-N-(but-2-yn-1-yl)-D-alaninate

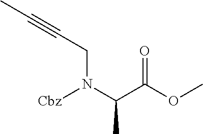 [SMILES: CC#CCN(C(=O)OCc1ccccc1)[C@H](C)C(=O)OC]

A 0° C. solution of 1-bromobut-2-yne (5.7 g, 43.1 mmol) and NaH (60%, 2 g, 51 mmol) in DMF (100 mL) was treated with methyl ((benzyloxy)carbonyl)-D-alaninate (9.3 g, 39.2 mmol). After 2 h, the resulting mixture was quenched with sat. aq. NH₄Cl and extracted with EtOAc. The organic layer was washed with brine, dried over Na₂SO₄, filtered and concentrated under reduced pressure to give the crude product with was purified by ISCO (0-10% EtOAc in PE) to afford the title compound (6.8 g, 60% yield) as a yellow oil. LCMS (m/z ES+): 290.3 (M+1).

### Step 2: N-((benzyloxy)carbonyl)-N-(but-2-yn-1-yl)-D-alanine

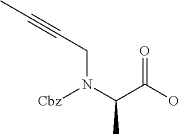 [SMILES: CC#CCN(C(=O)OCc1ccccc1)[C@H](C)C(=O)O]

A solution of methyl N-((benzyloxy)carbonyl)-N-(but-2-yn-1-yl)-D-alaninate (3.8 g, 13.1 mmol) in LiOH (33 mL, 6 N) and THF (66 mL) was stirred at ambient temperature. After 3 h. the resulting mixture was acidified with 3 N HCl and extracted with EtOAc. The organic layer was washed with brine, dried over Na₂SO₄, filtered and concentrated under reduced pressure to give the title compound (3.5 g, 97% yield) as a yellow oil which was used in the next step without further purification. LCMS (m/z ES−): 274.3 (M−1).

### Step 3: benzyl (R)-but-2-yn-1-yl(1-(methoxy(methyl)amino)-1-oxopropan-2-yl)carbamate

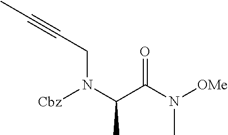 [SMILES: CC#CCN(C(=O)OCc1ccccc1)[C@H](C)C(=O)N(C)OC]

A mixture of N-((benzyloxy)carbonyl)-N-(but-2-yn-1-yl)-D-alanine (3.5 g, 12.7 mmol), N,O-dimethylhydroxylamine, HCl salt (1.6 g, 16.5 mmol), and DIPEA (3 mL) in DMF (30 mL) was treated with HBTU (7.4 g, 19.1 mmol). After 1 h, the resulting mixture was quenched with sat. aq. NaHCO₃ and extracted with EtOAc. The organic layer was washed with brine, dried over Na₂SO₄, filtered and concentrated under reduced pressure. The residue was purified by silica gel chromatography (0-15% EtOAc in PE) to afford the title compound (3.8 g, 95% yield) as a colorless oil. LCMS (m/z ES+): 319.0 (M+1).

### Step 4: benzyl (R)-but-2-yn-1-yl(1-oxopropan-2-yl)carbamate

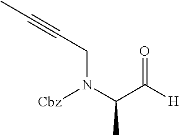 [SMILES: CC#CCN(C(=O)OCc1ccccc1)[C@H](C)C=O]

A −78° C. solution of benzyl (R)-but-2-yn-1-yl(1-(methoxy(methyl)amino)-1-oxopropan-2-yl)carbamate (3.8 g, 11.9 mmol) in THF (38 mL) was treated with DIBAL-H (16 mL, 23.8 mmol, 1.5M in toluene). After 4 h, the resulting mixture was quenched with 1N HCl and extracted with EtOAc. The organic layer was washed with brine, dried over Na₂SO₄, filtered and concentrated under reduced pressure. The residue was purified by silica gel chromatography (0-15% EtOAc in PE) to afford the title compound (2.5 g, 83% yield) as a colorless oil. ¹H NMR (400 MHz, CDCl₃) δ 9.63 (d, J=12.1 Hz, 1H), 7.43-7.28 (m, 5H), 5.17 (d, J=15.2 Hz, 2H), 4.26-4.06 (m, 3H), 1.78 (t, J=2.4 Hz, 3H), 1.45-1.37 (m, 3H). LCMS (m/z ES+): 260.5 (M+1).

### Step 5: benzyl (R)-but-2-yn-1-yl(but-3-yn-2-yl)carbamate

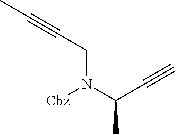 [SMILES: C#C[C@@H](C)N(CC#CC)C(=O)OCc1ccccc1]

A mixture of benzyl (R)-but-2-yn-1-yl(1-oxopropan-2-yl)carbamate (2.5 g, 9.7 mmol), dimethyl (1-diazo-2-oxopropyl)phosphonate (2.2 g, 11.6 mmol) and K₂CO₃ (2.7 g, 19.3 mmol) in MeOH (25 mL) was stirred ambient temperature. After 3 h, the resulting mixture was partitioned between EtOAc and H₂O. The organic layer was washed with sat. aq. NH₄Cl and brine, dried over Na₂SO₄, filtered and concentrated under reduced pressure. The residue was purified by silica gel chromatography (0-10% EtOAc in PE) to afford the title compound (1.0 g, 41% yield) as a colorless oil. LCMS (m/z ES+): 256.4 (M+1).

### Step 6: benzyl (R)-but-2-yn-1-yl(pent-3-yn-2-3/1)carbamate

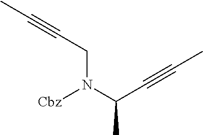 [SMILES: CC#CCN(C(=O)OCc1ccccc1)[C@H](C)C#CC]

A −20° C. solution of benzyl (R)-but-2-yn-1-yl(but-3-yn-2-yl)carbamate (600 mg, 2.34 mmol) in THF (6 mL) was treated with n-BuLi (2.5M, 1.87 mL, 4.69 mmol). After 1 h, Mel (0.43 mL, 7.03 mmol) was added and the reaction mixture was allowed to warm to ambient temperature over 1 h. The reaction mixture was quenched with sat. NH₄Cl aq. solution and extracted with EtOAc. The organic layer was washed with brine, dried over Na₂SO₄, filtered and concentrated under reduced pressure to give the crude product. The residue was purified by silica gel chromatography (0-10% EtOAc in PE) to afford the title compound (250 mg, 40% yield) as a colorless oil. ¹H NMR (400 MHz, CDCl₃) δ 7.44-7.27 (m, 5H), 5.18 (s, 2H), 5.12-5.03 (m, 1H), 4.18-4.04 (m, 2H), 1.91-1.67 (m, 6H), 1.43 (d, J=7.0 Hz, 3H). LCMS (m/z ES+): 270.3 (M+1).

### Step 7: (R)-benzyl 5-((S)-1-(tert-butoxy)-2-ethoxy-2-oxoethyl)-1,4,7-trimethyl-6-(trimethylsilyl)isoindoline-2-carboxylate

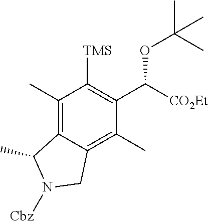 [SMILES: CCOC(=O)[C@@H](OC(C)(C)C)c1c(C)c2c(c(C)c1[Si](C)(C)C)[C@@H](C)N(C(=O)OCc1ccccc1)C2]

A mixture of [Rh(cod)]BF₄ (13.3 mg, 0.033 mmol), (R)-BINAP (20.4 mg, 0.033 mmol) in DCM (1 mL) was stirred at ambient temperature under a H₂ atmosphere for 4 h. The reaction mixture was charged with N₂ and a solution of ethyl (S)-2-(tert-butoxy)-4-(trimethylsilyl)but-3-ynoate (120 mg, 0.47 mmol) in DCM (1 mL) was introduced. After heated up to 40° C., a solution of benzyl (R)-but-2-yn-1-yl(pent-3-yn-2-yl)carbamate (250 mg, 0.94 mmol) in DCM (2 mL) was added dropwise to the reaction mixture and stirred at 40° C. for 30 min. The resulting mixture was concentrated under reduced pressure to give the crude product which was purified by silica gel chromatography (0-10% EtOAc in PE) to afford the title compound (150 mg, 61% yield) as a yellow oil and a 1:1 mixture of (R)-methyl regioisomers. LCMS (m/z ES+): 526.5 (M+1).

### Step 8: (R)-benzyl 5-((S)-1-(tert-butoxy)-2-ethoxy-2-oxoethyl)-6-iodo-1,4,7-trimethylisoindoline-2-carboxylate

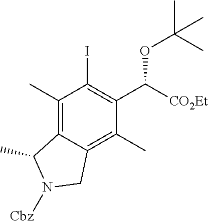 [SMILES: CCOC(=O)[C@@H](OC(C)(C)C)c1c(C)c2c(c(C)c1I)[C@@H](C)N(C(=O)OCc1ccccc1)C2]

A 0° C. solution of benzyl (R)-5-((S)-1-(tert-butoxy)-2-ethoxy-2-oxoethyl)-1,4,7-trimethyl-6-(trimethylsilyl)isoindoline-2-carboxylate (150 mg, 0.286 mmol) and NaHCO₃ (480 mg, 5.71 mmol) in DCM (4 mL) was treated with a solution of ICI (51 mg, 0.314 mmol) in DCM (0.3 mL). After 20 min, the resulting mixture was quenched with sat. aq. Na₂S₂O₃ and extracted with EtOAc. The organic layer was washed with brine, dried over Na₂SO₄, filtered and concentrated under reduced pressure. The residue was purified by silica gel chromatography (0-20% EtOAc in PE) to afford the title compound (153 mg, 93% yield) as a yellow oil and a 1:1 mixture of (R)-methyl regioisomers. LCMS (m/z ES+): 580.4 (M+1).

### Step 9: (1R,6R)-benzyl 5-((S)-1-(tert-butoxy)-2-ethoxy-2-oxoethyl)-6-(8-fluoro-5-methylchroman-6-yl)-1,4,7-trimethylisoindoline-2-carboxylate

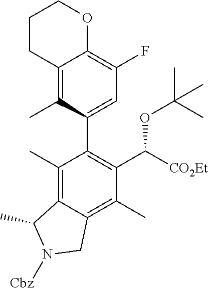

A mixture of benzyl (2S)(M)-5-(-1-(tert-butoxy)-2-ethoxy-2-oxoethyl)-6-iodo-1,4,7-trimethylisoindo line-2-carboxylate (153 mg, 0.264 mmol), (8-fluoro-5-methylchroman-6-yl)boronic acid (89 mg, 0.423 mmol), Pd₂(dba)₃ (48.4 mg, 0.053 mmol), MePhos (19.3 mg, 0.053 mmol) and K₃PO₄ (168 mg, 0.793 mmol) in DMF (2 mL) was stirred at 80° C. under an atmosphere of N₂. After 18 h, the reaction mixture was partitioned between EtOAc and H₂O. The organic layer was washed with sat. aq. NH₄Cl and brine, dried over Na₂SO₄, filtered and concentrated under reduced pressure. The residue was purified by silica gel chromatography (0-30% EtOAc in PE) to afford the title compound (80 mg, 50% yield) as a colorless oil and a 1:1 mixture of (R)-methyl regioisomers. LCMS (m/z ES+): 618.6 (M+1).

### Step 10: Ethyl (S)-2-(tert-butoxy)-2)-((1R,6R)-6-(8-fluoro-5-methylchroman-6-yl)-1,4,7-trimethylisoindolin-5-yl)acetate

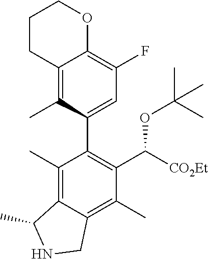

A mixture of benzyl (1R,6R)-5-((S)-1-(tert-butoxy)-2-ethoxy-2-oxoethyl)-6-(8-fluoro-5-methyl chroman-6-yl)-1,4,7-trimethylisoindoline-2-carboxylate (80 mg, 0.13 mmol) and 10% Pd/C (40 mg) in MeOH (2 mL) was hydrogenated under a H₂ atmosphere (1 atm). After 1 h, the reaction mixture was filtered through a pad of Celite and the residue was concentrated under reduced pressure to give the crude title product (60 mg, quant. yield) as a brown oil which was used in the next step without further purification. Product was a 1:1 mixture of (R)-methyl regioisomers LC/MS (m/z) ES+=484.1 (M+1).

### Step 11: Ethyl (S)-2-(tert-butoxy)-2-((1R,6R)-6-(8-fluoro-5-methylchroman-6-yl)-2-(3-fluorobenzoyl)-1,4,7-trimethylisoindolin-5-yl)acetate

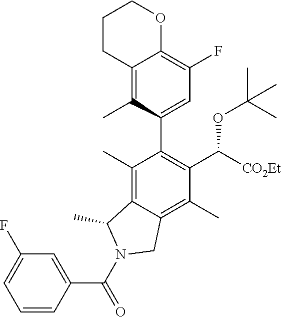

A solution of ethyl (S)-2-(tert-butoxy)-2-((1R,6R)-6-(8-fluoro-5-methylchroman-6-yl)-1,4,7-trimethylisoindolin-5-yl)acetate (60 mg) and 3-fluorobenzoic acid (32 mg, 0.25 mmol) in EtOAc (3 mL) was added propylphosphonic anhydride (150 mg, 0.25 mmol, 50% EtOAc solution) and Et₃N (0.11 mL, 0.74 mmol). After 1 h, the reaction mixture was quenched with sat. aq. NaHCO₃ and extracted with EtOAc. The organic layer was washed with brine, dried over Na₂SO₄, filtered and concentrated under reduced pressure. The residue was purified by silica gel chromatography (0-30% EtOAc in PE) to afford the title compound (47 mg, 60% yield) as a yellow oil and a 1:1 mixture of (R)-methyl regioisomers. LC/MS (m/z) ES+=606.5 (M+1).

### Step 11: (S)-2-(tert-butoxy)-2-((1R,6R)-6-(8-fluoro-5-methylchroman-6-yl)-2-(3-fluorobenzoyl)-1,4,7-trimethylisoindolin-5-yl)acetic acid

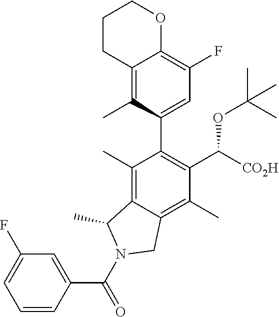

A solution of ethyl (S)-2-(tert-butoxy)-2-((1R,6R)-6-(8-fluoro-5-methylchroman-6-yl)-2-(3-fluorobenzoyl)-1,4,7-trimethylisoindolin-5-yl)acetate (47 mg, 0.078 mmol) in dioxane (4 mL) and H₂O (0.7 mL) was treated with LiOH (28 mg, 1.17 mmol) and heated to 80° C. After 18 h, the reaction mixture was cooled to ambient temperature and acidified with 1N HCl and extracted with EtOAc. The organics layer was washed with brine, dried over Na₂SO₄, filtered and concentrated under reduced pressure. The residue was purified by reverse phase HPLC (C18, 55-100% MeCN in H₂O with 0.1% formic acid) to afford the title compound (4.0 mg, 9% yield) as a white powder and a 1:1 mixture of (R)-methyl regioisomers. ¹H NMR (400 MHz, CDCl₃) δ 9.34 (br, 1H), 7.41-7.32 (m, 1H), 7.32-7.25 (m, 1H), 7.25-7.20 (m, 1H), 7.11 (dd, J=16.4, 7.9 Hz, 1H), 6.56 (d, J=11.5 Hz, 1H), 5.77-5.59 (m, 1H), 5.04-4.94 (m, 1H), 4.75 (d, J=14.7 Hz, 1H), 4.40 (d, J=14.5 Hz, 1H), 4.19 (dd, J=10.7, 6.0 Hz, 2H), 2.71-2.53 (m, 2H), 2.39-2.09 (m, 3H), 2.05 (dd, J=10.1, 5.9 Hz, 2H), 1.85-1.73 (m, 3H), 1.65-1.41 (m, 6H), 1.05 (d, J=4.7 Hz, 9H). LC/MS (m/z) ES+=578.6 (M+1).

### Anti-HIV Activity

### MT4 Assay

Antiviral HIV activity and cytotoxicity values for compounds of the invention from Table 1 were measured in parallel in the HTLV-1 transformed cell line MT-4 based on the method previously described (Hazen et al., 2007, In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV (Hazen et al., “In vitro antiviral activity of the novel, tyrosyl-based human immunodeficiency virus (HIV) type 1 protease inhibitor brecanavir (GW640385) in combination with other antiretrovirals and against a panel of protease inhibitor-resistant HIV”, Antimicrob. Agents Chemother. 2007, 51: 3147-3154; and Pauwels et al., “Sensitive and rapid assay on MT-4 cells for the detection of antiviral compounds against the AIDS virus”, J. of Virological Methods 1987, 16: 171-185).

Luciferase activity was measured 96 hours later by adding a cell titer glo (Promega, Madison, Wis.). Percent inhibition of cell protection data was plotted relative to no compound control. Under the same condition, cytotoxicity of the compounds was determined using cell titer Glo™ (Promega, Madison, Wis.). IC₅₀s were determined from a 10 point dose response curve using 3-4-fold serial dilution for each compound, which spans a concentration range >1000 fold.

These values are plotted against the molar compound concentrations using the standard four parameter logistic equation:

y=((Vmax*x\hat{ }n)/(K\hat{ }n+x\hat{ }n))+Y2
where:
Y2=minimum y n=slope factor
Vmax=maximum y x=compound concentration [M]
K=EC₅₀

When tested in the MT4 assay compounds were found to have IC₅₀ values listed in Table 1.

TABLE 1

| | IC50 |
|---|---|
| Example | (μM) |
| 1 | 0.0017 |

1. A compound of Formula I or a pharmaceutically acceptable salt thereof: 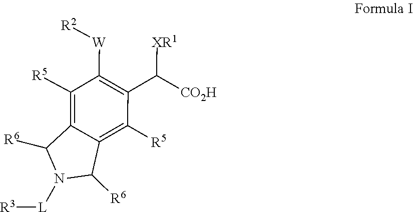
wherein:
X is O or CH₂;
R¹ is C₁₋₆alkyl wherein said alkyl may contain cycloalkyl portions;
W is a bond, —CH═CH—, —C≡C—, C₁₋₃alkylene, —CH₂C(O)NH—, —NHC(O)—, —N(CH₃)C(O)—, —N(CH₃)C(O)CH₂—, —C(O)—, —CH₂C(O)—, or —NHC(O)CH₂—, wherein each W is optionally substituted by 1 or 2 methyl groups;
R² is H, C₁₋₆alkyl, C₅₋₁₄aryl, C₃₋₇cycloalkyl, C₃₋₇cycloalkenyl, C₃₋₉heterocycle, or C₅₋₉heteroaryl, wherein each R² group is optionally substituted by one to four substituents selected from halo, C₁₋₆alkyl, C₁₋₆hetereoalkyl, or C₁₋₆alkylene or C₁₋₆hetereoalklylene wherein said C₁₋₆alkylene or C₁₋₆hetereoalklylene is bonded to adjacent carbon atoms on said C₅₋₁₄aryl, C₃₋₇cycloalkyl, C₃₋₇cycloalkenyl, C₃₋₉heterocycle, or C₅₋₉heteroaryl to form a fused ring;
L is a bond, —CH₂(CO)—, —C₁₋₃alkylene-, —SO₂—, —C(O)—, —C(S)—, —C(NH)—, —C(O)NH—, —C(O)NHCH₂—, —C(O)N—, —C(O)OCH₂—, —C(O)O—, —C(O)C(O)—, —SO₂—NH—, or —CH₂C(O)—;
R³ is H, CN, oxo, C₁₋₆alkyl, C₅₋₁₄aryl, CH₂C₅₋₁₄aryl, CH₂C₃₋₇cycloalkyl, C₃₋₇cycloalkyl, C₃₋₇spirocycloalkyl, C₃₋₇cycloalkenyl, C₃₋₉heterocycle, or C₅₋₉heteroaryl, or R³ may join together with an R⁶ to form a fused 5-7 membered ring, and wherein each R³ group is optionally substituted by one to four substituents selected from halo, oxo, C₁₋₆alkyl, C₃₋₇cycloalkyl, C₁₋₃fluoroalkyl, —OC₁₋₆alkyl, —C(O)R⁴, —C(O)NR⁴, —C(O)NHR⁴, C₅₋₁₄aryl, C₁₋₆hetereoalkyl, —B(OH)₂, C₃₋₉heterocycle, C₅₋₉heteroaryl, —C(O)OC₁₋₆alkyl, or two substituents may bond together to form a fused, spiro, or bridged ring and that fused, spiro, or bridged ring may optionally be substituted with R⁴;
R⁴ is CN, halo, —OC₁₋₆alkyl, C₁₋₆alkyl, C₃₋₇cycloalkyl, C₃₋₉heterocycle, or C₅₋₁₄aryl;
each R⁵ is independently H, C₁₋₃alkyl, C₃₋₆cycloalkyl, CH₂F, CHF₂, or CF₃;
each R⁶ is independently H, or C₁₋₃alkyl, C₅₋₁₄aryl, C₃₋₉heterocycle, C₅₋₉heteroaryl, —C(O)NR⁴, or —C(O)NHR⁴, or both R⁶ may together comprise 2-4 carbon atoms and join together to form a bridged ring system, with the proviso that at least one R⁶ is other than H;
and wherein each heterocycle, heteroaryl, heteroalkyl, and heteroalkylene comprises one to three heteroatoms selected from S, N, B, or O.

2. A compound or salt according to claim 1 wherein R¹ is C₁₋₆alkyl.

3. A compound or salt according to claim 1 wherein X is O.

4. A compound or salt according to claim 1 wherein W is a bond.

5. A compound or salt according to claim 1 wherein R² is optionally substituted phenyl.

6. A compound or salt according to claim 5 wherein R² is phenyl substituted by one to four substituents selected from fluorine, methyl, —CH₂CH₂CH₂O— wherein said —CH₂CH₂CH₂O— is bonded to adjacent carbon atoms on said phenyl to form a bicyclic ring, or —NHCH₂CH₂O— wherein said —NHCH₂CH₂O— is bonded to adjacent carbon atoms on said phenyl to form a bicyclic ring.

7. A compound or salt according to claim 1 wherein R³ is C₁₋₆alkyl, phenyl, naphthyl, cyclopentyl, cyclohexyl, pyridyl, or tetrahydropyranyl, each of which is optionally substituted by 1-3 substituents selected from halogen, C₁₋₆alkyl, —OC₁₋₆alkyl, C₁₋₃fluoroalkyl, or phenyl.

8. A compound or salt according to claim 1 wherein each R⁵ is methyl.

9. A compound or salt according to claim 1 wherein the stereochemistry on the carbon to which XR¹ is bound is as depicted below.

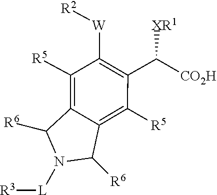

10. (canceled)

11. A pharmaceutical composition comprising a compound or salt according to claim 1.

12. A method for treating a viral infection in a patient mediated at least in part by a virus in the retrovirus family of viruses, comprising administering to said patient a composition according to claim 11.

13. The method of claim 12 wherein said viral infection is mediated by the HIV virus.

14-16. (canceled)
